(19) United States
(12) Patent Application Publication
(10) Pub. No.: US 2016/0143802 A1
(43) Pub. Date: May 26, 2016

(54) MEDICAL DEVICE

(71) Applicant: PROMOITALIA GROUP S.P.A., POZZUOLI-NA (IT)

(72) Inventors: Egidio Tranfaglia, Napoli (IT); Matanò Valerio, Napoli (IT)

(21) Appl. No.: 14/437,185

(22) PCT Filed: Oct. 25, 2012
(86) PCT No.: PCT/IT2012/000332

§ 371 Date: Apr. 20, 2015

(51) Int. Cl. A61H 9/00; A61F 7/00

(52) U.S. Cl. CPC A61H 9/0057; A61F 7/00; A61F 2007/0087; A61F 2007/0093

(57) ABSTRACT

A device for the treatment of pathologies related to accumulation of visceral fat and for the treatment of EFP in all its stages, comprising ‘clever’ handles for generation of acoustic waves, vacuum-assisted suction and cooling.

### BACKGROUND OF THE INVENTION

1. Field of the Invention

The present invention refers to a medical device for the treatment of pathologies related to accumulation of fat in a subject's tissue portion, in particular for the treatment of pathologies related to accumulation of visceral fat and for the treatment of EFP (edematous-fibrosclerotic panniculopathy) in all its stages.

2. Description of the Prior Art

The use of acoustic-wave and cryotherapy devices in aesthetic medicine and in cosmetics is per se known in the state of the art.

However, acoustic-wave devices present on the market in aesthetic medicine, owing to the shape of their handle, do not allow targeted treatments and this can cause damage to surrounding tissues or ineffective treatments.

Moreover, the dangerousness of state-of-the-art devices is also highlighted, since because of electrode wear, rather fast at high powers, there is no precision control on the energy.

Moreover, cryotherapy devices existing on the market utilize, in the portion into contact with the skin, materials oft-times causing cold burns.

Overall, therefore, the features of the devices present in aesthetic medicine which use acoustic waves or cryotherapy are too aggressive, or ineffective, or, unlike the invention proposed herein, are not capable of treating both adiposity and EFP.

Moreover, the devices available on the market have ranges that are very wide, or too deep, and oft-times also eat into healthy tissues, causing numerous side effects. In other cases, the ranges used prove to be simply ineffective for EFP and adiposity.

Therefore, object of the present invention is to solve the problems still left open by the known art, and this is attained by a medical device as defined in claim 1.

Further features of the device of the invention at issue are defined in the corresponding dependent claims.

### a. SUMMARY OF THE INVENTION

The present invention, by overcoming the problems of the known art, entails several evident advantages.

In particular, the present invention allows the combined use of plural technologies, specifically acoustic waves and cryotherapy, in case associated also to a suction.

Specifically, cryotherapy induces adipocyte apoptosis and acoustic waves carry out a mechanical action on the fibrous components of EFP. Moreover, the alternation of thermal action of cold induced by cryotherapy (capable of inducing vasoconstriction) and of the mechanical action of acoustic waves (capable of inducing vasodilation), produces a vascular gymnastics capable of improving venous stasis, a physiopathological cause of EFP.

The device according to the present invention allows a dynamic field unique in its kind and unites in a single apparatus the advantages of the generation of acoustic waves, those of vacuum (suction) and of cryolipolysis.

The device according to the present invention is adjusted automatically, guaranteeing patient safety and avoiding burns and scalds typical of traditional devices, in which the continuous emission of mechanical or acoustic waves used generates such a heat as to cause scalds, burns, or, in the best of cases, highly bothersome and not at all comfortable treatments.

In fact, on the device a microcontroller is fitted which immediately stops the machine and sends on the display an alarm signal in case it detects that the skin or the handle is beginning to overheat.

The peculiar point-like shape of the transducer, together with the specific parameters, of the device subject-matter of this invention, enables to localise the application even in very superficial pathologies such as edematous EFP and adiposity.

Moreover, the geometry of the handle enables a right focal distance also for obese patients requiring a greater pressure in the focus.

With this clinically suitable focus, it is also avoided the resorting in obese patients to greater pressure on the skin, which is one of the main factors causing pain in this type of treatment.

Moreover, the handle delivering acoustic waves has a wide contact surface and a wide entry angle, minimizing pain for the patient.

Oft-times, moreover, the handles of devices present on the market suddenly stop working due to wear, causing significant discomfort to operators.

The present invention overcomes this drawback by the inserting of a control function for controlling the total impulses (or “blows”) counter, allowing to check the impulses performed and stop machine operation upon reaching a predetermined number of impulses performed, e.g. 1000000. Upon reaching this limit the handle must be replaced or regenerated.

Moreover, the device according to the present invention provides a contact region of the cryotherapy handle, made of ceramized aluminum, so as to avoid any risk of cold burn.

The device provides a programmable central unit, allowing to set the correct operating sequences, with the best combination of values of intensity and of frequency and temperature for the pathologies to be treated.

The emissions (of electro-mechanical energy) delivered by the acoustic-wave handle, manipulated by the operator, help to fight EFP in all its stages, and localised adiposities. It is fundamental to point out that during the treatment blood vessels, lymphatic system and nerves undergo no alteration, owing to the selectivity of the (adipose tissue-specific) waves used.

### BRIEF DESCRIPTION OF THE DRAWINGS

These and other advantages, as well as the features and the operation modes of the present invention, will be made apparent in the following detailed description of preferred embodiments thereof, given by way of a non-limiting example. Reference will be made to the figures of the annexed drawings, wherein:

FIG. 1 is a perspective view of a medical device according to the present invention;

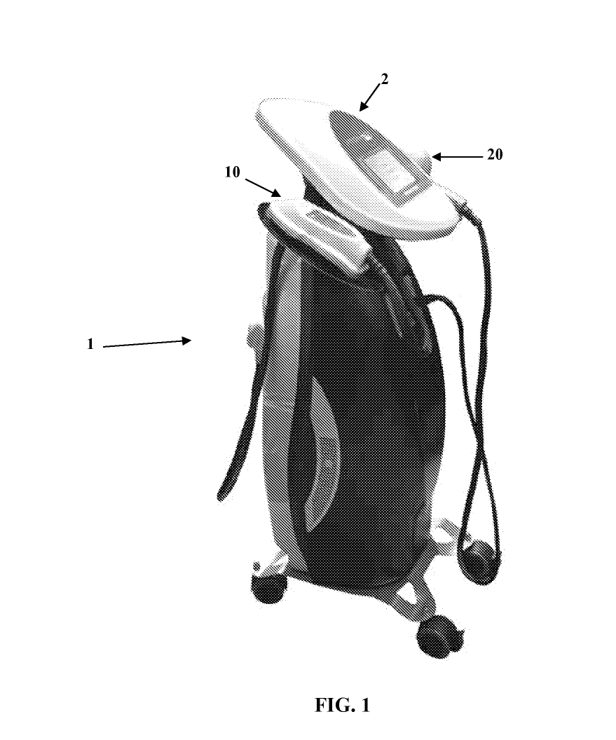

FIGS. 2A and 2B are views of a first handle according to the present invention; and

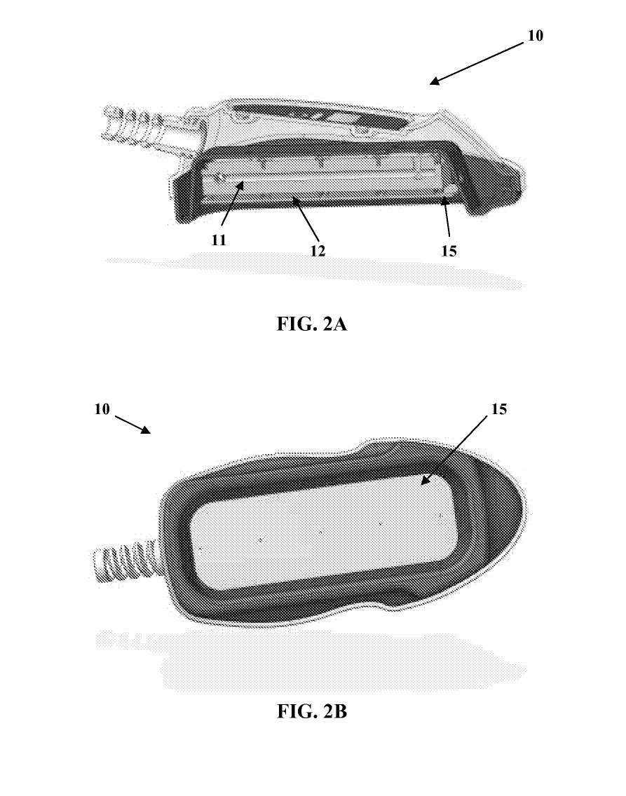

FIGS. 3A and 3B are views of a second handle according to the present invention.

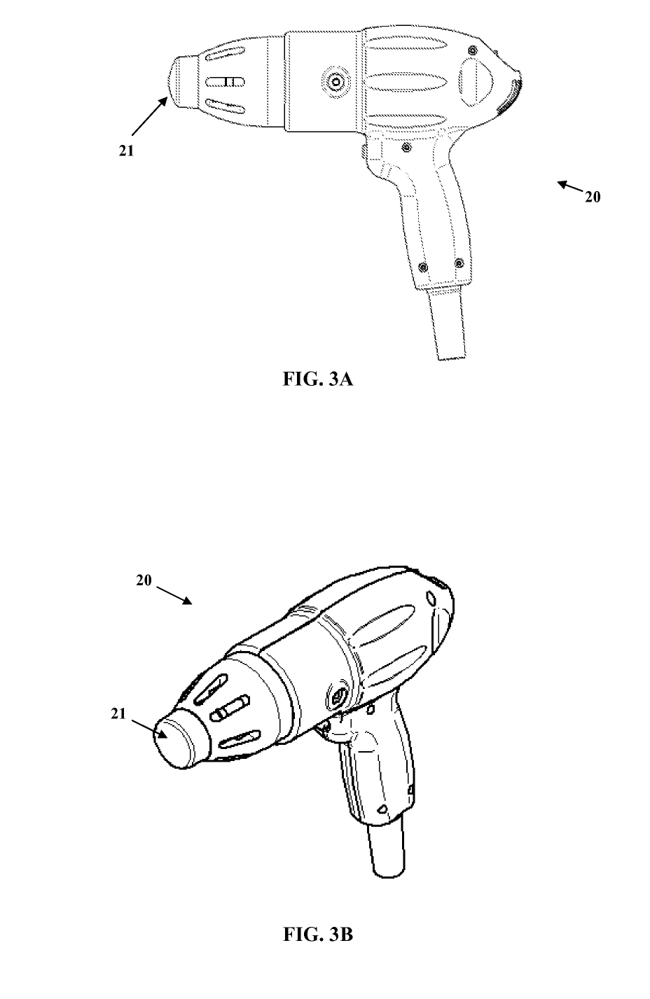

The present invention will be described in detail hereinafter, making reference to the above-indicated figures.

### a. DETAILED DESCRIPTION OF REPRESENTATIVE EMBODIMENTS

In general, a medical device 1 according to the present invention comprises a programmable central unit 2, apt to drive at least one handle 10, 20.

Moreover, the device provides first means for the thermal conditioning of a tissue portion of the subject to be treated and second means for the mechanical stressing of such tissue portion.

According to an embodiment, the device further comprises third means for the generation of a negative pressure at such tissue portion.

Advantageously, the programmable unit 2 is programmed for operating such first, second and third means according to predefined sequences and respective intensity values.

According to an embodiment of the present invention, the device provides a first handle 10.

Such first handle is preset for accommodating the above-indicated first means for the thermal conditioning. The first handle 10 could accommodate also the third means, where present, for the generation of a negative vacuum pressure at the tissue portion to be treated.

The first means for the thermal conditioning of said tissue portion comprises at least one element apt to exchange heat with the external environment, specifically, a dissipator. Moreover, such means comprises at least one Peltier cell, driven by the central unit, and a contact element for contacting the tissue portion to be treated, preferably made of ceramized aluminum. Overall, the handle is such that the contact element can thermally condition the tissue portion so as to be able to reach temperatures comprised in the range between −6° C. and +5° C., preferably between −5° C. and +2° C.

Advantageously, a handle cooling system may be provided. Such a system may be air- or fluid-assisted, or of hybrid type.

According to the preferred embodiment, the thermal conditioning means comprises a series of two or more Peltier cells.

The same handle 10 can possibly accommodate also means for generating a vacuum pressure at the zone to be treated.

This since the zone to be treated may be prepared by means of a joint action of cryotherapy and suction, prior to a subsequent mechanical action, to obtain a synergistic result and alleviate pain of a subsequent treatment.

To this end, the third means for the generation of a negative vacuum pressure is apt to generate a vacuum pressure comprised between 0 and −290 mbar. Such a pressure guarantees a correct positioning and contact of the cryotherapy handle to the skin surface. Moreover, the range of vacuum effect performs an action of separating and evenly distributing fatty masses, for an easier disposal of excess adipose cells (adipocytes).

According to an embodiment, the present invention further comprises a second handle 20, preset for accommodating the above-indicated second means for the mechanical stressing of the tissue portion to be treated.

According to the preferred embodiment, such mechanical stressing means comprises at least one generator of acoustic waves, preferably of electromagnetic type, e.g. piezoelectric, and a transducer for transmitting such acoustic waves to said tissue portion.

Advantageously, the transducer has a point-shaped contact region for contact with the tissue portion.

Generated shock waves are preferably of impulsive type. In the following table the main features of such acoustic waves are reported, features which are all advantageously programmable through the central unit.

Acoustic waves table

| | |
|---|---|
| Emission power output on the handle | 25 W (max) |
| Power adjustment (pressure impulses) | Adjustable from 100 to 500 |
| | bar/cm² |
| Energy output (J) | 500 = 0.327 J |
| | 450 = 0.258 J |
| | 400 = 0.169 J |
| | 350 = 0.057 J |
| | 300 = 0.031 J |
| | 250 = 0.014 J |
| | 200 = 0.006 J |
| | 150 = 0.004 J |
| | 100 = 0.002 J |
| Pressure output (bar) | 500 = 8.17 bar |
| | 450 = 6.45 bar |
| | 400 = 4.21 bar |
| | 350 = 1.42 bar |
| | 300 = 0.78 bar |
| | 250 = 0.36 bar |
| | 200 = 0.15 bar |
| | 150 = 0.09 bar |
| | 100 = 0.04 bar |
| Frequency | between 1 and 40 Hz |

The central unit allows the programming and setting of all parameters of the device, and of the operating sequences selected for each pathology to be treated.

Advantageously, a touch-screen-controlled microprocessor enables the operator to use preset programs in a simple way, by following operation steps on the board display.

Therefore, preferably, the central unit provides “driven” programs; by following them, the operator does not have to set the values of the single emission parameters, and therefore needs no specific training in the use of the apparatus. Of course, the operator could always adopt a “free” operation mode, in which he/she will have the freedom to set each parameter of the device according to his/her own decisions and needs.

The firmware will be implemented on a microcontroller of the Freescale family, series HCS12, and implemented on dedicated hardware.

Its main functions are:
1) Management of a user interface by means of a 320×240 pixel LCD with touch-screen.
2) Management of screen shots in various languages or by selection carried out by the user.
3) Management of dedicated default parameters for 3 typologies of treatments.
4) Management of operation parameter settings.
5) Management of actuation of a Peltier cell apt to cool the body part on which the suitable handle is positioned.
6) Management of an open-loop proportional electrovalve, in order to provide a vacuum level sufficient to keep the suitable handle adhered to the body part on which it is applied.
7) Management of a dual-coil electromagnet for actuating vibrations on the body part to which the suitable handle is applied.
8) Management of a timer indicating the treatment time, settable by the user, with acoustic signaling when the time is up and interruption of actuations.
9) Management of a total and partial counter of “blows” performed by the vibrations of the vibration handle.
10) Management of resetting to zero of the partial counter of “blows” performed.
11) Management of vibration handle replacement on the basis of the total number of “blows” performed.
12) Management of a watch-dog apt to reset uP in case of undesired stop of the program.
13) Management of removal of at least 1 handle
14) Management of maximum temperature of the vibration handle
15) Management of a base parameter configuration menu accessible by password.

The device, as already indicated a few times, provides the setting of parameters such as: Operation time, Vacuum temperature and pressure; for the cryotherapy-delivering handle: Operation time, Power and Number of Repeats for the acoustic waves-delivering handle. Such parameters have been determined in specific protocols which vary according to zone and pathology to be treated. Such values are modifiable by the operator.

By way of example, hereinafter indications will be given with regard to some of the treatable pathologies, specifically:
excess localized adiposity
Stage I and II EFP
Stage III and IV EFP

### Parameters Related to Thermal Conditioning.

Temperature.

Corresponds to the temperature that the handle may reach in order to perform a correct cryotherapy, i.e. capable of inducing adipose cell apoptosis. Such value ranges from +2° C. to −5° C.

The lower the Temperature, the more effective the freezing ability, and therefore adipose cell apoptosis, for the treatment of excess visceral adiposity. When instead the temperature is higher, and greater than 0° C. (+2° C.), cooling ability will be such as to induce a peripheral vasoconstriction, capable of assisting in a vascular gymnastics effective in EFP treatment, without inducing apoptosis.

It is pointed out that in EFP, regardless of its clinical stage, the first cause of the physiopathological process inducing its formation is venous stasis. Therefore, in case of cellulite it is envisaged the use of temperatures higher than 0° C.

Thus, for the treatment of excess visceral adiposity the setting of a temperature lower than 0° C. (−5° C.) was established, in order to ensure a cold-induced apoptosis process.

Operation Time.

It is the time, expressed in minutes, needed to treat a certain area. Such parameter was set on the basis both of the size of the area to be treated and the type of pathology. In particular, it should be taken into account that in case of excess localised adiposity longer cryotherapy times have to be set, required for apoptosis of the treated area; instead, in case of EFP shorter times are used. However, a distinction should be drawn between stage I and stage II EFP, in which times no longer than 5 minutes are envisaged, to be alternated with wave therapy, and stage III- and stage IV EFP, in which times no longer than 15 minutes are suggested, to be alternated with wave therapy. Such an alternation has the purpose of triggering the so-called vascular gymnastics induced by cold-induced vasoconstriction, cryotherapy and mechanical wave-induced vasodilation.

Pressure.

Indicates the vacuum pressure value associated to the handle, in order to guarantee a correct positioning and contact of the cryotherapy handle to the skin surface. Moreover, the range of the effect of the vacuum singled out performs the action of separating and evenly distributing fatty masses, for an easier disposal of excess adipose cells.

The value ranges from a minimum of 22 to a maximum of 100. Pressure used is inversely proportional to tissue thickness. The greater the thickness, the smaller the vacuum value to be set will be.

### Parameters for Acoustic Wave-Delivering Handle

Power.

Power is expressed in Bar, or in Kg/cm², and ranges from 100 to 500 Bar. As to power used, it is pointed out that it is proportional to tissue thickness and type of pathology to be treated.

In the following table, ranges of preferred values are indicated. However, it is to be understood that such values should not be understood as exclusive, and therefore limiting the present invention.

| THICKNESS OF ZONE | POWER |
|---|---|
| TO BE TREATED | Pressure impulses |
| <1-2 cm | 100 |
| 2-3 cm | 150-200 |
| 3-4 cm | 200 |
| 4-5 cm | 250-300 |
| 5-6 cm | 350 |
| 6-7 cm | 350-400 |
| 7-8 cm | 400-450 |

The higher the POWER, the deeper the penetration of the acoustic wave in skin tissue. The lower the POWER, the more superficial the penetration in skin tissue. However, considering that EFP, regardless of its stage, does not exhibit a particularly deep localisation, a range of not overly high power values (e.g., 100-150) was established; instead, for the treatment of adiposities reference is made to the related table.

Operation Time.

It is the time, expressed in minutes, needed to treat a certain area.

Number of Repeats.

Indicates the number of times/second in which the acoustic wave is repeated; from this, also the emission rate thereof is deduced. Said parameter ranges, e.g., from 0.1 to 16 Hz. In the treatment of adiposities, a not overly high number of repeats is used, whereas in the treatment of EFP higher frequencies are used, to induce mechanical vasodilation.

From the above it is deduced that tissues characterized by the presence of excess localized adiposity require the use of a cryotherapy handle with high times of use (at least 30 minutes) and temperatures lower than 0, which thanks to the prolonged action of an intense cold will have greater impact on a tissue, inducing adipocyte apoptosis. In that case the use of the acoustic wave-delivering handle will be at low powers and repeats, and would serve above all to potentiate the effect of any lipolytic product. Instead, tissues affected by EFP at any stage require short-time use of a cryotherapy handle and temperatures higher than 0° C., in order to induce the sole vasoconstriction, whereas the action of the shock wave handle, owing to a greater impact (as used at greater powers and repeats) will act incisively on the fibrous component of EFP and on the vascular component, thanks to a mechanical action of vasodilation.

The protocols of use, developed on the basis of the above considerations, are reported hereinafter.

The precise range of the values of pressure and delivered energy of such invention, singled out by the scientific staff of the Inventor company, enables to avoid acoustic wave applications causing painful, harmful or ineffective treatments.

### Protocol for Excess Localised Adiposity

Use of ice-cold handle for carrying out cryotherapy for a time ranging from 20 to 40 minutes, with:
Slow motions on the zone (area) in a radial-pattern sliding mode
−5° C. temperature

Thereafter, on the same area, use of the shock wave-delivering handle in association with a conductive gel for 10-15 minutes.
Variable powers (150-350) to be varied in connection with tissue depth, tissue consistency and the patient's subjective sensitivity.
Variable number of repeats, in connection with fat typology (e.g. frequency of 5-8).

4-6 sessions every 15 days are suggested.

### Protocol for Stage I-II EFP

Use of ice-cold handle for carrying out 5 minutes of cryotherapy (no apoptosis, but vascular gymnastics)
Slow motions on the zone in a sliding mode
Temperature in the neighborhood of 0° C.

Use of shock wave-delivering handle for carrying out 5 minutes of therapy.
Powers are lower (100-150)
Variable number of repeats, in connection with capillary fragility, tissue thickness, patient's subjective sensitivity and extent of the edema (frequencies equal to 03-05).

It is suggested to alternate the use of the two handles for at least 4 times (20 minutes per side). 4-6 sessions every 7-10 days are suggested.

### Protocol for Stage III-IV EFP

Use of cryotherapy-delivering handle for 15 minutes
Slow motions on the zone in a sliding mode
Temperature equal to −1° C.

Use of acoustic wave-delivering handle for carrying out 10-15 minutes of therapy.
Powers are lower (100-150)
The number of repeats is higher with respect to the treatment of Stage I and II EFP, but anyhow variable in connection with capillary fragility, tissue thickness, patient's subjective sensitivity (frequencies equal to 06-11).

Total time per area equal to 25-30 minutes.

4-6 sessions every 7-10 days are suggested.

It is underlined that such protocols can be modified by the medical operator prior to the treatment, after having performed an accurate visit on the patient in order to introduce any modification to the protocols suggested. The number of sessions may vary on the basis of the volume of the zone (area) to be treated, with an interval of 15 days between sessions for treatment of localised adiposity and of 7-10 days for treatment of EFP in the various stages.

The present invention has hereto been described with reference to preferred embodiments thereof. It is understood that other embodiments might exist, all falling within the concept of the same invention, and all comprised within the protective scope of the claims hereinafter.

1. A medical device (1) for the treatment of pathologies related to an accumulation of fat in a subject's tissue portion, comprising:
a programmable central unit (2), apt to drive at least one handle (10, 20);
first means for the thermal conditioning of said tissue portion;
second means for the mechanical stressing of said tissue portion;
wherein said programmable unit (2) is programmed for operating said first and second means according to predefined sequences and respective intensity values.

2. The device according to claim 1, further comprising third means for the generation of a negative pressure at said tissue portion, operable by said central unit (2) according to predefined sequences and intensity values.

3. The device (1) according to claim 1, wherein said at least one handle (10, 20) comprises a first handle (10) preset for accommodating said first means for the thermal conditioning.

4. The device (1) according to claim 3, wherein said first handle (10) is further preset for accommodating said third means for the generation of a negative pressure.

5. The device (1) according to claim 1, wherein said at least one handle (10, 20) comprises a second handle (20) preset for accommodating said second means for the mechanical stressing.

6. The device (1) according to claim 1, wherein said first means for the thermal conditioning of said tissue portion comprises at least one heat exchanging element apt to exchange heat with the external environment.

7. The device (1) according to claim 6, wherein said heat exchanging element comprises a dissipator (11).

8. The device (1) according to claim 6, wherein said heat exchanging element comprises at least one Peltier cell (12), driven by said central unit (2).

9. The device (1) according to claim 1, wherein said first means for the thermal conditioning of said tissue portion is apt to reach temperatures comprised between −6° C. and +5° C.

10. The device (1) according to claim 1, wherein said first means for the thermal conditioning of said tissue portion comprises a contact element (15) for contacting with said tissue portion, said element being made of ceramized aluminum.

11. The device (1) according to claim 1, wherein said second means for the mechanical stressing of said tissue portion comprises at least one generator of acoustic waves and a transducer (21) for transmitting such waves to said tissue portion.

12. The device (1) according to claim 11, wherein said acoustic waves are of impulsive type, such that each impulse have a programmable length comprised between 1 and 10 ms and be such as to exert on said tissue a programmable pressure comprised between 100 and 500 bar/cm².

13. The device (1) according to claim 12, wherein said impulses be generated at a programmable frequency comprised between 1 and 40 Hz.

14. The device (1) according to claim 11, wherein said transducer (21) has a point-shaped contact region for contact with said tissue portion.

15. The device (1) according to claim 2, wherein said third means for the production of a negative pressure is apt to generate a vacuum pressure comprised between 0 and −290 mbar.
